(19) United States
(12) Patent Application Publication
(10) Pub. No.: US 2006/0149093 A1
(43) Pub. Date: Jul. 6, 2006

(54) Process for saparating a hydroxybenzonitrile-type compound

(76) Inventors: Jean-Francis Spindler, Lyon (FR); Roland Jacquot, Francheville (FR); Michel Alas, Melle (FR)

Correspondence Address: Jean-Louis Seugnet;Rhodia Inc, Intellectual Property Dept, 259 Prospect Plains Road CN 7500, Cranbury, NJ, 08512-7500, US

(21) Appl. No.: 10/311,100

(22) PCT Filed: Jun. 13, 2001
(86) PCT No.: PCT/FR01/01833

§ 371 Date: Jun. 10, 2003

(30) Foreign Application Priority Data
Jun. 14, 2000 (FR) 00/07568

(51) Int. Cl. C07C 255/50

(52) U.S. Cl. 558/425

(57) ABSTRACT

The invention concerns a method for separating a hydroxybenzonitrile-type compound obtained by an amination/dehydration process. More particularly, the invention concerns 2-hydroxybenzonitrile, also called 2-cyanophenol. The inventive method for separating a hydroxybenzonitrile compound from a reaction gas flow comprising it wholly or partly in the form of an ammonium salt is characterized in that it consists in displacing the ammonium ions, through a physical treatment carried out on the reaction gas flow derived from its preparation or from the solid previously recuperated from said flow or according to a chemical treatment carried out on the reaction gas flow, after liquefaction.

The present invention relates to a process for separating a hydroxybenzonitrile type compound obtained by means of an amination/dehydration process. More particularly, it relates to 2-hydroxybenzonitrile, also known as 2-cyanophenol. The invention also relates to a process for separating and purifying a hydroxybenzonitrile type compound contained in a gaseous reaction stream.

Hydroxybenzonitriles are products of major industrial interest as they are used as colorants and as intermediates in the preparation of active ingredients such as herbicides, fungicides and insecticides.

One route to obtaining hydroxybenzonitriles consists of aminating an alkyl hydroxybenzoate followed by dehydration.

More particularly, German patent DE-A-2 020 866 describes a process for preparing 4-hydroxybenzonitrile in the gas phase by reacting ammonia and the methyl ester of 4-hydroxybenzoic acid in the presence of a catalyst, namely phosphoric acid deposited on a support such as silica gel.

A process of the same type has been described in French patent FR-A-2 332 261 and Canadian patent CA-A-2 177 939, except that the catalyst is respectively boron phosphate or boron phosphate doped with a transition metal from group Va, VIa, IIb, IIIb of the periodic table published in the Bulletin de la Societe Chimique de France, n^{o} 1 (1966).

The problem when separating the hydroxybenzonitrile type compound obtained using that type of process, which consists of reacting methyl 2-hydroxybenzoate and ammonia in the gas phase in the presence of a dehydration catalyst, is that difficulties arise because the product obtained solidifies as soon as it condenses, which makes isolation difficult.

Other difficulties reside in the formation of by-products due to two types of secondary reactions.

The higher the temperature of the reaction medium, the faster the product obtained trimerizes to S-triazine. It has been found that from 140° C., trimerization is immediate and exhibits an auto-catalytic nature.

A further type of secondary reaction that occurs is hydrolysis of the 2-hydroxybenzonitrile obtained to 2-hydroxybenzamide due to the water liberated during preparation of the 2-hydroxybenzonitrile, which reaction is accentuated by the presence of ammonia.

Thus, there is a genuine industrial problem in obtaining 2-hydroxybenzonitrile from the gaseous reaction stream containing it.

We have now discovered, and this constitutes the subject matter of the present invention, a process for separating a hydroxybenzonitrile type compound from a gaseous reaction stream containing it either completely or partially in the form of an ammonium salt, characterized in that it consists, with the aim of obtaining a hydroxybenzonitrile type compound, of displacing the ammonium ions:
by means of a physical treatment carried out on the gaseous reaction stream deriving from its preparation or by means of a physical treatment of the solid already recovered from the gaseous reaction stream after liquefaction, or of said same solid in solution;
or by means of a chemical treatment carried out on the gaseous reaction stream after liquefaction.

The term “hydroxybenzonitrile type compound” as used throughout the description of the present invention means an aromatic compound carrying at least one hydroxyl group and a nitrile group and/or the hydrated form of said compound, i.e., the nitrile group is completely or partially replaced by an amide group.

In accordance with a first implementation of the present invention, the ammonium ions are displaced using a physical treatment, more precisely a heat treatment.

In accordance with a second implementation of the present invention, the ammonium ions are displaced using a chemical treatment, more precisely an acid treatment.

It has been discovered that the hydroxybenzonitrile type compound is present in the gaseous reaction stream in a form in which the OH group is in the salt form, i.e., it is in the form of the ammonium salt, and that the free OH content represents less than 10 mole %, preferably less than 5 mole %.

In order to separate the hydroxybenzonitrile type compound, it has been discovered that the ammonium ions have to be displaced.

At the end of the vapour phase reaction for preparing the hydroxybenzonitrile type compound by means of amination/dehydration of an alkyl hydroxybenzoate type compound, the gaseous reaction stream is at a high temperature, generally more than 200° C., preferably in the range 200° C. to 600° C., more preferably in the range 350° C. to 450° C.

In accordance with the process of the invention, it has been discovered that it is possible to obtain a benzonitrile type compound with a free OH group by heat treating a compound in the salt form without aiding secondary reactions such as trimerization and hydrolyzing the nitrile group.

In a first variation of the process of the invention, the gas stream is heat treated.

In a second variation, the process of the invention consists of liquefying the gaseous reaction stream then recovering the hydroxybenzonitrile type compound in its salt form as a solid from said stream and heat treating the solid product obtained either directly or after dissolving.

In a further variation, the process of the invention consists of liquefying the gaseous reaction stream then treating it with an acid resulting in the production of a 2-hydroxybenzonitrile type compound then recovering it in an organic phase which is separated out.

The process of the invention is applicable to any aromatic compound containing at least one aromatic ring containing 6 carbon atoms and carrying at least one nitrile group and an OH group which is completely or partially in its salt form.

The invention encompasses benzene rings, naphthalene rings or a concatenation of benzene rings separated by a covalent bond, an alkyl or alkylidene group containing 1 to 4 carbon atoms, or an atom (for example oxygen or a functional group such as CO).

More particularly, the invention concerns compounds that can be represented as follows:

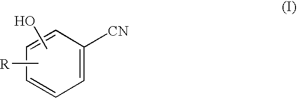

in which formula R represents one or more substituents.

The hydroxybenzonitrile with formula (I) can carry one or more substituents provided that they do not interfere with the process of the invention.

The number of substituents present on the ring depends on the carbon condensation of the cycle and on the presence or otherwise of unsaturated bonds in the cycle.

The maximum number of substituents that can be carried by a cycle can readily be determined by the skilled person.

In the present text, the term “plurality” generally means less than 4 substituents on an aromatic nucleus.

Examples of substituents are given below; this list is not limiting in nature. The following can be cited:
linear or branched alkyl groups preferably containing 1 to 6 carbon atoms, more preferably 1 to 4 carbon atoms;
linear or branched alkenyl groups, preferably containing 2 to 6 carbon atoms, more preferably 2 to 4 carbon atoms;
linear or branched halogenoalkyl groups, preferably containing 1 to 6 carbon atoms and 1 to 13 halogen atoms, more preferably 1 to 4 carbon atoms and 1 to 9 halogen atoms;
the hydroxyl group;
the NO₂ group;
R₁—O— alkoxy or R₁—S— thioether groups in which R₁ represents a linear or branched alkyl group containing 1 to 6 carbon atoms, more preferably 1 to 4 carbon atoms or the phenyl group; or alkenyloxy groups, preferably an allyloxy group;
R₁—CO—O— acyloxy or aroyloxy groups in which the group R₁ has the meanings given above;
R₁—CO— acyl or aroyl groups, in which group R₁ has the meanings given above;
a halogen atom, preferably a fluorine atom;
a CF₃ group.

The invention is of particular application to separating and purifying 4-hydroxybenzonitrile and 2-hydroxybenzonitrile.

In the following description of the present invention, for simplification, the process of the invention will be described in its application to the preparation of 2-hydroxybenzonitrile, but it can be applied to all of the compounds cited above.

In accordance with the process of the invention, we start from a gaseous reaction stream (F1) essentially comprising 2-hydroxybenzonitrile, either completely or partially in its salt form. This means that the quantity of 2-hydroxybenzonitrile expressed as a mole % with respect to the alkyl 2-hydroxybenzoate employed is at least 50%, preferably at least 75% and still more preferably in the range 80% to 95%.

The gas stream (F1) also comprises ammonia in an amount of 200 to 600 mole %, nitrogen in an amount of 5 to 200 mole %, the alcohol liberated by the starting ester, usually methanol in an amount of 50 to 100 mole %, and the water formed during the reaction, about 50 to 100%.

The gaseous reaction stream (F1) can optionally comprise 0 to 5 mole % of phenolic compounds, for example phenol, 0 to 5 mole % of starting hydroxybenzoate ester, and products resulting from secondary reactions such as hydrolysis or trimerization, in particular 0 to 10 mole % of 2-hydroxybenzamide optionally N-alkylated by the liberated alcohol, for example N-methyl-(2-hydroxybenzamide), and less than 3 mole % of S-triazine.

The different concentrations of the components of the stream are given by way of illustration and are in no way limiting in nature.

It should be noted that this reaction stream is essentially gaseous but the invention also encompasses the case of an aerosol, i.e., a portion of the gaseous stream may be condensed so that liquid particles are present, preferably in an amount of less than 10% by volume.

This gaseous reaction stream can derive from an alkyl hydroxybenzoate amination/dehydration reaction.

It can be represented by the following formula:

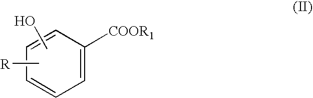

in which formula, R has the meaning given above in formula (I) and R₁ generally represents an alkyl group containing 1 to 4 carbon atoms, preferably a methyl or ethyl group.

Preferred hydroxybenzoates that can be employed that can be cited are methyl 2-hydroxybenzoate, ethyl 2-hydroxybenzoate and ethyl 4-hydroxybenzoate. Preferably, methyl or ethyl 2-hydroxybenzoates are used.

Thus, the compound with formula (II) is reacted with ammonia in the vapour phase in the presence of a heterogeneous catalyst.

Examples of catalysts that can be cited are boron phosphate, optionally doped as mentioned in CA-A-2 177 939, or phosphoric acid deposited on a support which may be silica and/or alumina and/or titanium oxide. Preferably, a silica gel or kieselguhr type support is used; the phosphoric acid represents 50% to 75% by weight of the catalyst.

While the quantity of ammonia engaged can equal the theoretical quantity determined by the stoichiometry of the reaction (i.e., one mole of ammonia per mole of hydroxybenzoate), amination is preferably carried out using an excess of ammonia. In general, it is preferable to use at least two moles, more particularly 2 to 5 moles of ammonia per mole of hydroxybenzoate. The excess ammonia present in the gas stream resulting from the amination reaction can be recycled.

The apparent dwell time for the gas stream with the catalyst, defined as the time in seconds during which one unit volume of gas mixture (measured under normal temperature and pressure) is in contact with one apparent unit volume of catalyst, can be in the range 0.001 sec to 10 min, preferably in the range 0.01 sec to 2 min.

The reaction is generally carried out at atmospheric pressure, at a temperature of 200° C. to 600° C.; preferably, it is in the range 350° C. to 450° C.

At the end of the reaction, a gaseous reaction stream (F1) is obtained comprising light gases, ammonia, water and an alcohol deriving from the starting alkyl hydroxybenzoate (preferably methanol) and heavy gases, essentially 2-hydroxybenzonitrile, which is completely or partially in its salt form, and the by-products mentioned above.

The process of the invention, which can produce a hydroxybenzonitrile type compound with a free OH group from a benzonitrile type compound, completely or partially in the form of the ammonium salt, can also be applied to a benzonitrile type compound in the hydrated form, i.e., a hydroxybenzamide type compound with formula (I) in which the CN group is replaced by a CONH₂ group, completely or partially in the ammonium salt form.

The scope of the present invention also encompasses applying the process of the invention to a hydroxybenzonitrile type compound in its hydrated form.

Said compound is obtained in a known manner by means of amination of an alkyl hydroxybenzoate type compound by reaction with ammonia in the presence of a conventional catalyst: glass beads, silica or silica-alumina.

### Physical Treatment of A reaction Stream Comprising 2-hydroxybenzonitrile in its Salt Form

In accordance with the process of the invention, it has been discovered that to separate 2-hydroxybenzonitrile from a gaseous reaction stream, the ammonium salt has to be displaced.

To this end, the temperature of the hot reaction stream is reduced to a temperature so that a liquid phase essentially comprising 2-hydroxybenzonitrile in its salt form is condensed, enabling decomposition of the ammonium salt leading to the liberation of ammonia.

In a first variation of the process of the invention, this operation is accomplished by bringing the gaseous reaction stream into contact with a solvent (organic solvent and/or water) then cooling the ensemble to a temperature below 200° C., preferably in the range 200° C. to 100° C., to obtain a liquid phase essentially comprising 2-hydroxybenzonitrile.

The gas stream is brought into the presence of a solvent (S1).

Several criteria govern the choice of solvent.

Firstly, the solvent has to be stable under the reaction conditions.

Secondly, the solvent must be vaporized in the selected temperature zone, i.e., its boiling point must be less than 250° C.

Advantageously, the solvent has a boiling point in the range 100° C. to 250° C., preferably in the range 100° C. to 200° C.

In a preferred variation, the solvent is selected that can also purify the 2-hydroxybenzonitrile obtained, by crystallization. Thus, it is a good solvent when hot but a poor solvent when cold, usually at ambient temperature, preferably in the range 15° C. to 25° C.

A polar or apolar solvent is used.

Examples of suitable solvents that can be cited are water, water/alcohol mixtures, for example methanol or ethanol; halogenated or non halogenated aromatic hydrocarbons, for example toluene, xylenes, ethylbenzene, monochlorobenzene; and ethers, for example anisole or 2-ethylhexanol.

When using water/alcohol mixtures, they generally contain less than 50% of alcohol, preferably 1% to 50%, more preferably 1% to 25% of alcohol and 50% to 99%, preferably 75% to 99% of water.

The quantity of solvent employed represents 30% to 500% of the weight of the gaseous reaction stream.

In practice, the hot gas stream is brought into contact with a co-current of the vaporized reaction solvent.

Light gases (F2) comprising ammonia, alcohol (usually methanol), water and in some cases an azeotropic water/organic solvent mixture are recovered, along with the heavy compounds (F3) essentially comprising 2-hydroxybenzonitrile and the solvent (organic solvent and/or water) and minor products such as phenolic compounds, for example phenol, the starting hydroxybenzoate ester, 2-hydroxybenzamide and possibly N-methyl-(2-hydroxybenzamide), and S-triazine.

The invention also concerns a process for separating and purifying 2-hydroxybenzonitrile.

The first step is carried out as described above and the heavy fraction (F3) essentially comprising 2-hydroxybenzonitrile and solvent is treated.

Purification is then carried out by distilling the products with the lower boiling points then distilling the 2-hydroxybenzonitrile.

The first fraction recovered between 20° C. and 40° C. at 0.4 mbars of pressure corresponds to the solvent.

The second fraction obtained between 40° C. and 110° C. at 0.4 mbars of pressure corresponds to phenol and the starting hydroxybenzoate.

The third fraction obtained at 110° C. under 0.4 mbars of pressure is constituted by 2-hydroxybenzonitrile.

The salicylamide, N-methylsalicylamide and S-triazine are recovered from the bottom.

The purity of the distilled 2-hydroxybenzonitrile is at least 95%, preferably at least 98%.

A further purification technique consists of crystallizing the 2-hydroxybenzonitrile then carrying out a solid/liquid separation of the crystallized product.

We start with the fraction (F3) from the separation step.

Then cooling to a temperature in the range 100° C. to −10° C., preferably in the range 10° C. to 0° C., causes the 2-hydroxybenzonitrile to crystallize.

The crystalline product is then separated using conventional solid/liquid separation techniques, preferably by filtration.

A solid is recovered that is essentially 2-hydroxybenzonitrile and a liquid phase (F6) comprising the solvent, a little 2-hydroxybenzonitrile (less than 5% by weight) and by-products.

The solid can be dried at a temperature in the range 30° C. to 80° C., preferably in the range 40° C. to 50° C.

The purity of the 2-hydroxybenzonitrile is very high, at over 90%, preferably more than 95%.

A further purification technique that can be employed is purification by refining.

We start from the fraction (F3) deriving from the separation step.

Firstly, it is cooled to a temperature in the range 100° C. to 30° C.

An aqueous phase (F7) and an organic phase (F8) are obtained that are separated after decanting.

The organic phase is essentially constituted by 2-hydroxybenzonitrile and solvent (S1).

The water and/or solvent is/are separated by distillation at a temperature of 90° C. to 100° C. and under a reduced pressure in the range 1 mbar to 1 bar.

A crude reaction mass is obtained that essentially comprises 2-hydroxybenzonitrile.

A refining purification operation is then carried out.

Refining is carried out discontinuously using apparatus that can carry out liquid/solid separation (dewatering, zone melting) and of a size that depends on the volume to be treated and on the number of devices employed. Further, the choice of type of apparatus is not critical. As an example, conventional draining sieves can be used, or other refining apparatus, for example those sold under the trade name PROAPT (registered trade mark). Vertical cylindrical tube exchanger type draining sieves could be used, for example.

The fraction (F9) is treated in one or more pieces of apparatus essentially by means of the following 4 phases:
phase 1 corresponds to slow crystallization of the charged mixture;
phase 2 corresponds to cold dewatering of the eutectic (mixture of 2-hydroxybenzonitrile+impurities such as the starting hydroxybenzoate, phenol, salicylamide and N-methylsalicylamide);
phase 3 corresponds to hot dewatering recovered during the reheating phase until the desired purity is obtained;
phase 4 corresponds to fusion-recovery of the product with the desired purity.

The production of fractions with substantially constant compositions facilitates automisation of this refining step.

Fraction (F9) comprising 2-hydroxybenzonitrile is sent to one or more pieces of refining apparatus.

Prior to phase 1, the apparatus is heated above the melting point of 2-hydroxybenzonitrile (98° C.), for example to between 100° C. and 120° C.

During phase 1, the mass is cooled, for example to a temperature in the range 10° C. to 50° C., over several hours, for example 5 to 15 h, which induces slow crystallization of the charged mixture.

After phase 1, the product remains liquid and is removed from the apparatus (phase 2) before passing to phase 3.

Phase 3 consists of slowly reheating the refining apparatus, possibly commenced during phase 2, for example to a temperature in the range 94° C. to 98° C., over several hours, for example 8 to 15 h.

The end of phase 3, which determines the purity of the product, can be determined either by measuring the crystallization point or by any other physico-chemical analytical technique.

Phase 4 requires heating the apparatus to a temperature of over 95° C. to melt the 2-hydroxybenzonitrile which is extracted in the molten state (F11).

The eutectic fractions (F10) recovered during refining can be recycled as a mixture or separately with the hot dewatering, preferably to the preceding step.

2-hydroxybenzonitrile is obtained with a purity of at least 98%, preferably at least 99%.

### Physical Treatment of Ammonium Cyanophenate

In a further implementation of the invention, 2-hydroxybenzonitrile is separated from the gaseous reaction stream (F1) containing it in the salt form, in a variation of the process that comprises the steps of liquefaction of the gaseous reaction stream, crystallization of the 2-hydroxybenzonitrile as an ammonium salt hereinafter termed “ammonium cyanophenate”, solid/liquid separation of the crystallized product then heat treatment of the separated solid to liberate ammonia, water, and an alcohol (preferably methanol) and to recover the 2-hydroxybenzonitrile.

In a further variation, the invention consists of heat treating the ammonium cyanophenate not in the solid form, but heat treating the ammonium cyanophenate in solution in an organic solvent (S2).

The gaseous reaction stream is then liquefied and the ammonium cyanophenate is crystallized and solid/liquid separation is carried out then the solid product obtained is dissolved in an organic solvent, preferably a polar solvent and finally, the organic solution obtained is heat treated.

More precisely, the gaseous reaction stream (F1) is first liquefied by cooling from 90° C.-100° C. to a temperature of 40° C.-30° C. and bringing it into contact with water, which can eliminate the light products (ammonia and water) and recover an aqueous phase comprising ammonium cyanophenate and ammonia.

The quantity of water employed represents 50% to 100% by weight of (F1).

In the next step, in a first step, the ammonium cyanophenate is crystallized.

By cooling the liquefied stream (F12) to a temperature in the range 0° C.-30° C., the ammonium cyanophenate is caused to crystallize.

The crystalline product is then separated using conventional solid/liquid separation techniques, preferably by filtering.

Separation is carried out at a temperature in the range 0° C. to 20° C.

A solid is recovered that is essentially ammonium cyanophenate and a liquid phase (F13) comprising the solvent, a little of the 2-hydroxybenzonitrile (less than 5% by weight) and by-products.

The recovered product can be represented by formula (III):

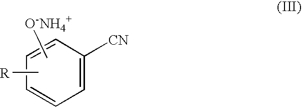

in which formula, R has the meanings given above.

The heat treatment is then carried out at a temperature in the range 80° C. to 20° C., and at a pressure in the range 1 mbar to 1 bar, preferably between 1 mbar and 500 mbar.

A light fraction (F14) is eliminated which comprises ammonia and the solvent (S1), and 2-hydroxybenzonitrile is recovered as a solid.

In a further variation of the invention, the heat treatment is carried out on the ammonium cyanophenate which is dissolved in an organic solvent (S2).

Firstly, the solid ammonium cyanophenate obtained as described above is dissolved by liquefying the gaseous stream, crystallization and separation resulting in ammonium cyanophenate in the solid form and a liquid phase (F15).

The ammonium cyanophenate is dissolved by adding a polar aprotic solvent.

Examples of suitable solvents that can be mentioned are dimethylformamide and N-methylpyrrolidone.

Preferably, dimethylformamide is used.

The quantity of solvent employed represents 30% to 500% of the weight of the ammonium cyanophenate.

An organic solution of ammonium cyanophenate is obtained that is then heat treated at a temperature in the range 80° C. to 20° C. and at a pressure in the range 1 mbar to 1 bar, preferably between 1 mbar and 500 mbar.

A light fraction (F16) comprising ammonia and the solvent is eliminated and a fraction (F17) is recovered which is 2-hydroxybenzonitrile in solution in a solvent (S2), and it is possible to isolate it conventionally by distillation or by cooling in a flaker.

### Chemical Treatment of a Stream Comprising 2-hydroxybenzonitrile in the Salt Form

In a further implementation of the invention, the 2-hydroxybenzonitrile is separated from the gaseous reaction stream (F1) containing it in its salt form, by chemically displacing the ammonium ions.

This variation of the process consists of liquefying the gaseous reaction stream, treating it with an acid to obtain the 2-hydroxybenzonitrile, and carrying out liquid/liquid separation to recover the 2-hydroxybenzonitrile in the organic phase.

More precisely, firstly the gaseous reaction stream (F1) is liquefied by cooling from 90° C.-100° C. to a temperature of 40° C.-30° C. and bringing it into contact with water to eliminate the light compounds (ammonia and water) and to recover an aqueous phase comprising ammonium cyanophenate and ammonia.

The quantity of water employed represents 50% to 700% of the weight of (F1).

The ammonium ions are then neutralized using an acid.

A Brönsted acid with a pKa, measured in water, of less than 6, preferably in the range −1 to 4, is used.

More particular examples that can be cited are sulphuric acid, hydrochloric acid, phosphoric acid and acetic acid.

The concentration of the acid solution is immaterial: it can be between 30% and 100%.

The quantity of acid employed is such that the pH obtained is between 6 and 1, preferably between 4 and 3.

The neutralization operation is carried out at a temperature that is advantageously in the range 90° C. to 30° C.

The reaction mixture obtained is then decanted to separate an aqueous phase (F19) comprising excess acid and ammonium salts, from an organic phase (F20) comprising the 2-hydroxybenzonitrile.

This latter can be purified using purification techniques such as distillation, crystallization and refining, as described above.

The present invention also concerns facilities for carrying out the different variation of the process of the invention.

### Physical Treatment of a Reaction Stream Comprising 2-hydroxybenzonitrile in the Salt Form

The present invention also concerns a facility for carrying out the process of the invention which comprises at least one unit for separating 2-hydroxybenzonitrile from a gaseous reaction stream and optionally, a 2-hydroxybenzonitrile purification unit.

The separation unit comprises:
a first column (1) intended to bring the gaseous reaction stream and the solvent (water and/or organic solvent) into contact;
a second column (2) the inlet to which is connected to the bottom of column (1), said column (2) being intended to product a light fraction comprising ammonia, water and an alcohol (preferably methanol) from the column head and from the column bottom, a heavy fraction essentially comprising 2-hydroxybenzonitrile, solvent (water and/or organic solvent), traces of ammonia and minor quantities of secondary products.

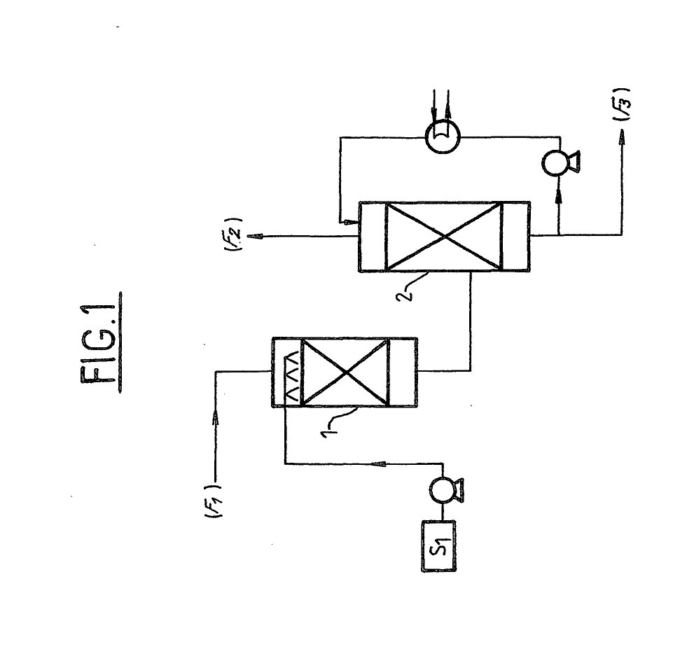

FIG. 1 shows a diagram of a separation unit in accordance with the invention.

The facility for separation comprises a first column (1) for ensuring good gas/liquid contact, for example a wash tower. It comprises means at the column head for admitting a gaseous reaction stream (F1) to be treated and means for admitting solvent (S1), for example a pump and a device (nozzles) for vaporizing the latter. In its lower portion, the column comprises means for evacuating a heavy phase (liquid and/or gas) connected to a second column (2) which is supplied at mid-height.

Column (2) for gas/liquid separation is a column comprising cooling means (condenser). Its upper portion is provided with means for withdrawing a gas phase comprising light compounds (F2) and means for withdrawing a liquid phase (F3).

In a preferred implementation of the invention, the facility comprises a separation unit and a purification unit.

The separation unit is completed by means ensuring purification of 2-hydroxybenzonitrile contained in the heavy fraction (F3) recovered at the outlet from the separation unit.

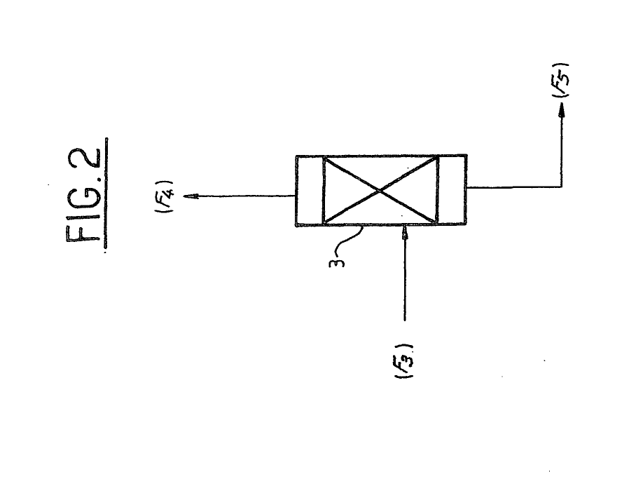

FIGS. 2 to 4 illustrate different means for carrying this out.

In a first preferred embodiment, which consists of carrying out the purification by distillation (FIG. 2), the facility also comprises a distillation column (3) supplied with the heavy fraction (F3) deriving from the preceding column (2) and designed to obtain:
at the column head, different fractions at a pressure of 0.4 mbar:
   light fractions with a boiling point of less than 40° C. at atmospheric pressure;
   a fraction between 40° C. and 110° C. corresponding to phenol and to methyl salicylate;
   a fraction at 110° C. corresponding to 2-hydroxybenzonitrile (F4);
and at the column bottom, different impurities (F5) with a boiling point of more than 110° C.: salicylamide, N-methylsalicylamide and S-triazine.

From this information, the skilled person will be capable of selecting the means to be employed as a function of the starting mixture. The following is simply an outline. The size (in particular the diameter) of the distillation columns depends on the circulating stream and on the internal pressure. They are dimensioned principally according to the flow rate of the mixture to be treated. The internal parameter, which is the number of theoretical stages, is determined in particular by the composition (ratios) of the entering mixture and the purity or composition of the mixture that should be obtained at the head and bottom of the distillation column. The columns can be packed with an ordered packing, as is known to the skilled person. Once the facility has been determined, the skilled person will adjust the operating parameters of the columns.

The distillation column (3) can advantageously, but not in a limiting fashion, be a column with the following specifications:
number of theoretical stages: 5 to 40, preferably 10 to 30;
reflux ratio R in the range 1 to 10;
low residence time, preferably less than 30 minutes.

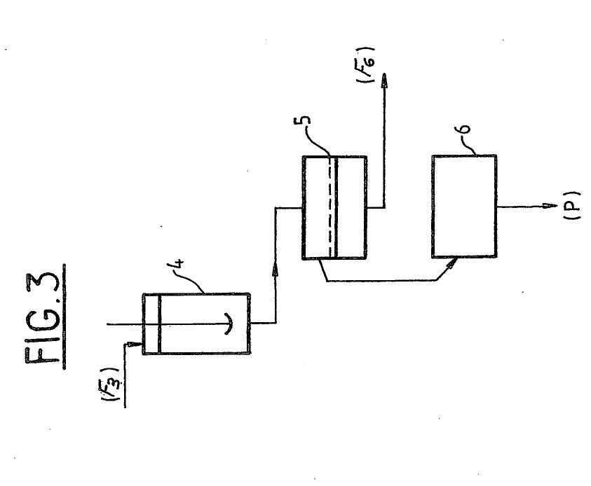

In a second embodiment, purification is accomplished by crystallization (FIG. 3). The facility comprises a crystallization reactor (4) provided with means for admitting the fraction (F3) and a withdrawal device, as is required for its operation. This reactor is provided with stirring means, and a temperature regulating device (hot/cold): cooling can be ensured by circulating cold water in a jacket or by means of an internal packing (coil).

It is connected to a solid/liquid separation device (5) such as an aspirator, agitated filter, filter-press, or multi-tube filter.

It can recover a solid essentially constituted by 2-hydroxybenzonitrile and an aqueous phase (F6) comprising the solvent, a little 2-hydroxybenzonitrile (less than 5% by weight) and by-products.

The separation device (5) can optionally be associated with drying means (6) such as a rotary drier or an agitated drier, enabling the 2-hydroxybenzonitrile to be recovered in the form of a powder (P).

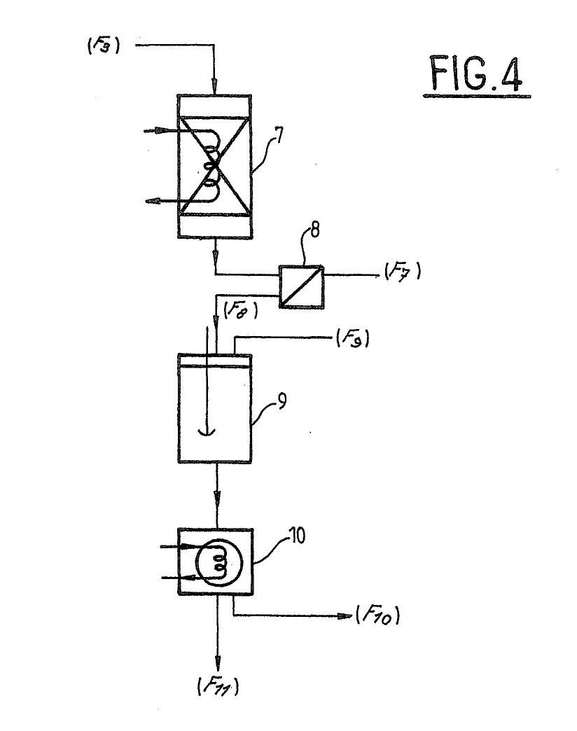

In a third embodiment (FIG. 4), which consists of carrying out purification by refining, the facility comprises a cooling tower (7) provided with means for admitting the fraction (F3) and a withdrawal device, as is necessary for its operation.

It is connected to a liquid/liquid separation device (8) such as a settler.

It can separate an aqueous phase (F7) from an organic phase essentially constituted by 2-hydroxybenzonitrile (F8).

This organic phase is directed to a reactor (9) provided with stirring means at a temperature regulating device (hot/cold). It is surmounted by a distillation column and provided with a reduced pressurization system that can separate the organic solvent and/or remaining water by distillation (F9).

The organic phase (F8) is routed to an apparatus (10) for refining (dewatering, zone melting) and dimensioned as a function of the volume to be treated and their number. The choice of apparatus type is also not critical. It may, for example, be a conventional draining sieve or other refining apparatus, for example those sold under the trade name PROAPT (registered trade mark). As an example, it is possible to use draining sieves of the vertical cylindrical tube exchanger type.

Different fractions are recovered containing impurities (F10) and a 2-hydroxybenzonitrile fraction (F11).

### Physical Treatment of Ammonium Cyanophenate

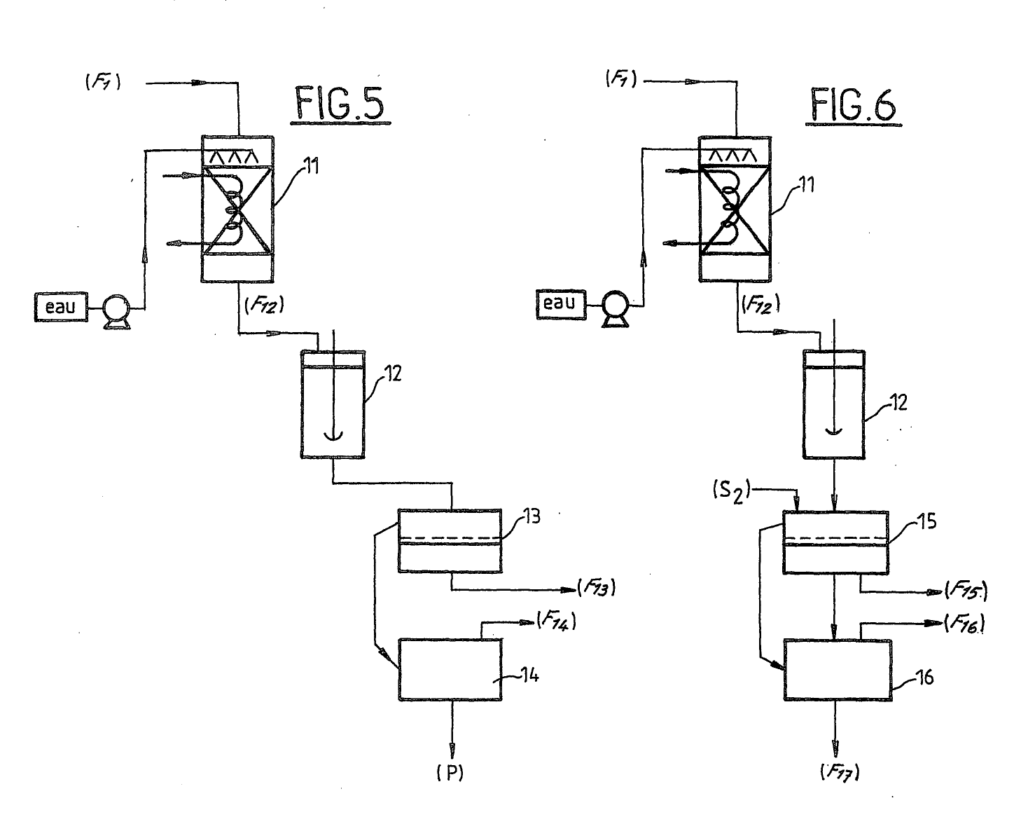

In accordance with a further variation of the invention, ammonium cyanophenate rather than the gaseous reaction stream comprising 2-hydroxybenzonitrile is heat treated, and the process is carried out in a facility comprising the separation unit illustrated in FIG. 5 or 6.

The facility shown in FIG. 5 comprises a wash tower (11) provided with a system for admitting gas (F1) and a device for introducing water via a pump that can recover from the bottom a liquid phase (F12) comprising water, crude ammonium cyanophenate and ammonia; the mixture is transferred to reactor (12).

In reactor (12) the ammonium cyanophenate is crystallized; it is provided with means for admitting the fraction (F12); said reactor is provided with agitation means and with a temperature regulating device (hot/cold).

In its lower portion it has a withdrawal device that can connect it to a solid/liquid separation device (13) such as an aspirator, agitated filter, filter-press or multi-tube filter.

It can recover a solid essentially constituted by ammonium cyanophenate and an aqueous phase (F13) comprising the solvent, a little 2-hydroxybenzonitrile (less than 5% by weight) and by-products.

The separation unit is associated with drying means (14) such as an agitated drier in which it undergoes a temperature and pressure cycle to decompose the solid ammonium cyanophenate to 2-hydroxybenzonitrile, allowing the 2-hydroxybenzonitrile to be recovered in the form of a powder (P) while eliminating the solvent and remaining ammonia (F14).

In a further variation shown in FIG. 6, the facility comprises, as in FIG. 5, a wash tower (11) and a reactor (12) in which the ammonium cyanophenate is crystallized and which is provided with means for admitting the fraction (F12); said reactor is provided with agitation means, and with a temperature regulating device (hot/cold).

Its lower portion comprises a withdrawal device that can connect it to a solid/liquid separation device (15) such as an aspirator, agitated filter, filter-press or multi-tube filter and is provided with a means for admitting a solvent (S2) to dissolve the ammonium cyanophenate.

It can recover the ammonium cyanophenate in an organic solution and an aqueous phase (F18) comprising the solvent (S1), a little 2-hydroxybenzonitrile (less than 5% by weight) and by-products.

The mixture is sent to a reactor (16) provided with stirring means, a temperature regulating device (hot/cold) surmounted by a distillation column and provided with a reduced pressurization system to separate the organic solvent and/or remaining water (F16) and to carry out thermal decomposition of the ammonium cyanophenate to 2-hydroxybenzonitrile by the joint application of temperature and pressure.

(F17) is recovered, namely 2-hydroxybenzonitrile in solution in a solvent (S2) and it is possible to isolated it conventionally by distillation or by cooling on a flaker.

### Chemical Treatment of Stream Comprising 2-Hydroxybenzonitrile in the Salt Form

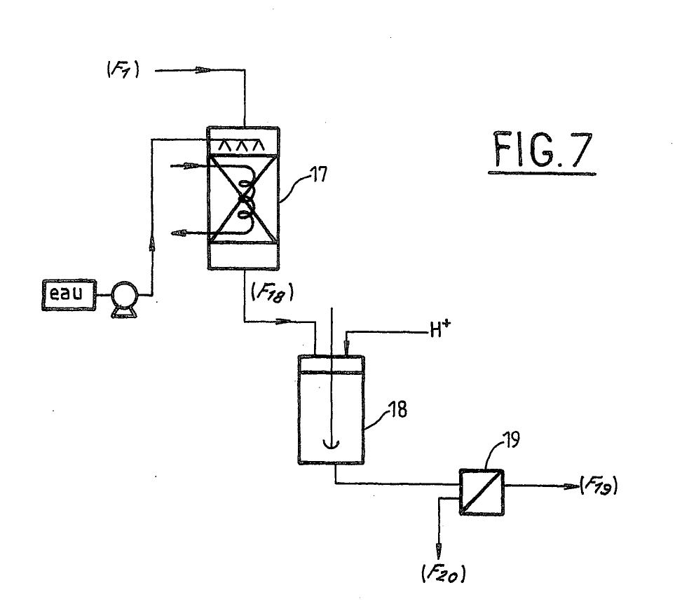

The separation unit shown in FIG. 7 comprises:
a wash tower (17) provided with a system for admitting gas (F1) and a device for introducing water via a pump that can recover a liquid phase (F18) comprising water, crude ammonium cyanophenate and ammonia from the bottom, which is transferred to a reactor (18);
a neutralization reactor (18) provided with agitation means, a temperature regulating device (hot/cold), a reactant introduction device (acid) and a pH regulation device (electrode): said reactor comprising a withdrawal device in its lower portion for connecting it to a device (19);
a liquid/liquid separation device (19) such as a settler that can separate an aqueous phase (F19) comprising the excess acid and ammonium salts and an organic phase (F20) comprising 2-hydroxybenzonitrile.

This latter can be purified using purification techniques such as distillation, crystallization and refining illustrated in FIGS. 2 to 4.

The process of the invention can produce very pure 2-hydroxybenzonitrile.

The invention will now be described in more detail by means of implementations of the invention that are taken by way of non limiting example.

### EXAMPLE 1

### Chemical Treatment of a Stream Comprising 2-hydroxybenzonitrile in its Ammonium Salt Form in Accordance with FIG. 7

The crude reaction stream from a reactor comprising 0.52 moles of 2-hydroxybenzonitrile in its salt form (2-HBN) and 1.7 moles of ammonia (F1) was dissolved in 72 g of water at 60° C. in the wash tower (17).

The stream (F18) was then transferred to a stirred reactor (18) where 35.1 g of 98% sulphuric acid was added, keeping the temperature at 60° C.

The final pH had to be in the range 3 to 4 when sulphuric acid addition was complete.

The upper phase (F20), crude 2-hydroxybenzonitrile, was recovered after decanting at 60° C. in a settler (19).

The titer of the crude 2-hydroxybenzonitrile was 61% by weight.

The recovery yield was 96% (0.506 moles).

### EXAMPLE 2

In the apparatus shown in FIG. 3, the crude 2-hydroxybenzonitrile obtained in Example 1 was purified by crystallization at a temperature of 0° C. in toluene as the solvent.

The following results were obtained:
purity of 2-hydroxybenzonitrile=95%;
isolated yield=74%.

### EXAMPLE 3

In the apparatus shown in FIG. 3, the crude 2-hydroxybenzonitrile obtained in Example I was purified by crystallization at a temperature of 0° C. in chlorobenzene as the solvent.

The following results were obtained:
purity of 2-hydroxybenzonitrile=99%;
isolated yield=55%.

### EXAMPLE 4

### Physical Treatment of Ammonium Cyanophenate According to FIG. 6

The crude reaction stream from a reactor comprising 0.52 moles of 2-hydroxybenzonitrile in its salt form (2-HBN) and 1.7 moles of ammonia (F1) was dissolved in 216 g of water at 60° C. in the wash tower (11).

The stream was charged into a reactor (12) where it underwent cooling to 15° C.

The precipitated ammonium 2-cyanophenate was recovered by filtering in a filter (15) where it was washed with 50 g of 5% by weight ammonia.

The precipitate was drained then re-dissolved in the filter in 70 g of dimethylformamide.

The ammonium 2-cyanophenate solution in dimethylformamide was transferred to reactor (16) where the water then the ammonia were eliminated by heating to 60° C. at 100-50 mbars.

The isolation yield of the 2-hydroxybenzonitrile was 72% and the purity (ex DMF) of the 2-hydroxybenzonitrile was 99.5%.

### EXAMPLE 5

### Physical Treatment of Reaction Stream Comprising 2-hydroxybenzonitrile in its Ammonium Salt Form, in Accordance with FIG. 1

The crude reaction stream from a reactor comprising 0.52 moles of 2-hydroxybenzonitrile in its salt form (2-HBN) and 1.7 moles of ammonia (F1) at 420° C. was brought into contact with in 200 g of xylene at in the wash tower (1).

The stream was then transferred to a column (2) where the mixture was cooled to 110° C. by an exchanger.

The ammonium 2-cyanophenate decomposed to ammonia and was eliminated from the column head.

The crude 2-hydroxybenzonitrile dissolved in xylene (F3) was recovered from the column bottom.

The tire of the crude 2-hydroxybenzonitrile was 89% by weight (ex solvent).

The recovery yield was 94% (0.49 moles).

### EXAMPLE 6

In the apparatus shown in FIG. 3, the crude 2-hydroxybenzonitrile obtained in Example 5 was purified by crystallization in xylene as the solvent.

The following results were obtained:
purity of 2-hydroxybenzonitrile=92%;
isolated yield=68%.

### EXAMPLE 7

### Physical Treatment of Reaction Stream Comprising 2-hydroxybenzonitrile in its Ammonium Salt Form, in Accordance with FIG. 1

The crude reaction stream from a reactor comprising 0.52 moles of 2-hydroxybenzonitrile in its salt form (2-HBN) and 1.7 moles of ammonia (F1) at 420° C. was brought into contact with 200 g of xylene in wash tower (1).

The stream was then transferred to a column (2) where the mixture was cooled to 110° C. by an exchanger.

The ammonium 2-cyanophenate decomposed to ammonia and was eliminated from the column head.

The crude 2-hydroxybenzonitrile dissolved in xylene (F3) was recovered from the column bottom.

The titer of the crude 2-hydroxybenzonitrile was 89% by weight (ex solvent).

The recovery yield was 94% (0.49 moles).

### EXAMPLE 8

In the apparatus shown in FIG. 3, the crude 2-hydroxybenzonitrile obtained in Example 7 was purified by crystallization in xylene as the solvent at 0° C.

The following results were obtained:
purity of 2-hydroxybenzonitrile=92%;
isolated yield=68%.

### EXAMPLE 9

In the apparatus shown in FIG. 3, the crude 2-hydroxybenzonitrile obtained in Example 7 was purified by crystallization at a temperature of −10° C. in a solvent which was a xylene/methanol mixture (95/5, w/w %).

The following results were obtained:
purity of 2-hydroxybenzonitrile=98%;
isolated yield=85%.

### EXAMPLE 10

### Physical Treatment of Reaction Stream Comprising 2-hydroxybenzonitrile in its Ammonium Salt Form, in Accordance with FIG. 1

The crude reaction stream from a reactor comprising 0.52 moles of 2-hydroxybenzonitrile in its salt form (2-HBN) and 1.7 moles of ammonia (F1) at 420° C. was brought into contact with 180 g of xylene and 20 g of DMF in the wash tower (1).

The stream was then transferred to a column (2) where the mixture was cooled to 110° C. by an exchanger.

The ammonium 2-hydroxybenzonitrile decomposed to ammonium and was eliminated from the column head.

The crude 2-hydroxybenzonitrile dissolved in the xylene+DMF (F3) was recovered from the column bottom.

The titer of the crude 2-hydroxybenzonitrile was 88% by weight (ex solvent).

The recovery yield was 97% (0.49 moles).

### EXAMPLE 11

In the apparatus shown in FIG. 3, the crude 2-hydroxybenzonitrile obtained in Example 7 was purified by crystallization at a temperature of −10° C. in a solvent which was a xylene/DMF mixture (90/10, w/w %).

The following results were obtained:
purity of 2-hydroxybenzonitrile=99%;
isolated yield=85%.

It should be understood that the invention defined in the accompanying claims is not limited to the particular implementations described in the above description but encompasses variations that do not depart from the spirit or the scope of the invention.

1. A process for separating a hydroxybenzonitrile type compound from a gaseous reaction stream containing it either completely or partially in the form of an ammonium salt, characterized in that it consists, with the aim of obtaining a hydroxybenzonitrile type compound, of displacing the ammonium ions:
by means of a physical treatment carried out on the gaseous reaction stream deriving from its preparation or by means of a physical treatment of the solid already recovered from the gaseous reaction stream after liquefaction, or of said same solid in solution;
or by means of a chemical treatment carried out on the gaseous reaction stream after liquefaction.

2. A process according to claim 1, characterized in that the hydroxybenzonitrile type compound has the following formula: 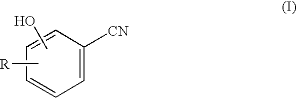
in which formula R represents one or more substituents.

3. A process according to claim 2, characterized in that the hydroxybenzonitrile type compound has formula (I) in which R is selected from:
linear or branched alkyl groups preferably containing 1 to 6 carbon atoms, more preferably 1 to 4 carbon atoms;
linear or branched alkenyl groups, preferably containing 2 to 6 carbon atoms, more preferably 2 to 4 carbon atoms;
linear or branched halogenoalkyl groups, preferably containing 1 to 6 carbon atoms and 1 to 13 halogen atoms, more preferably 1 to 4 carbon atoms and 1 to 9 halogen atoms;
the hydroxyl group;
the NO₂ group;
R₁—O— alkoxy or R₁—S— thioether groups in which R₁ represents a linear or branched alkyl group containing 1 to 6 carbon atoms, more preferably 1 to 4 carbon atoms or the phenyl group; or alkenyloxy groups, preferably an allyloxy group;
R₁—CO—O— acyloxy or aroyloxy groups in which the group R₁ has the meaning given above;
R₁—CO— acyl or aroyl groups, in which group R₁ has the meaning given above;
a halogen atom, preferably a fluorine atom;
a CF₃ group.

4. A process according to claim 1, characterized in that the hydroxybenzonitrile type compound is 4-hydroxybenzonitrile or 2-hydroxybenzonitrile.

5. A process according to claim 1, characterized in that the hydroxybenzonitrile type compound is wholly or partially in its hydrated form.

6. A process according to one claims 1 to 5, characterized in that the gaseous reaction stream (F1) essentially comprises 2-hydroxybenzonitrile, either completely or partially in its salt form; preferably, the quantity of 2-hydroxybenzonitrile, expressed as a mole % with respect to the alkyl 2-hydroxybenzoate employed, is at least 50%, preferably at least 75% and still more preferably in the range 80% to 95%.

7. A process according to claim 6, characterized in that gas stream (F1) also comprises ammonium in an amount of 200 to 600 mole %, nitrogen in an amount of 5 to 200 mole %, the alcohol liberated by the starting ester, preferably methanol in an amount of 50 to 100 mole %, water formed during the reaction, about 50 to 100%; optionally 0 to 5 mole % of phenolic compounds, preferably phenol, 0 to 5 mole % of the starting hydroxybenzoate ester, and products resulting from secondary reactions, such as hydrolysis or trimerization, in particular 0 to 10 mole % of 2-hydroxybenzamide optionally N-alkylated by the liberated alcohol, for example N-methyl-(2-hydroxybenzamide), and less than 3 mole % of S-triazine.

8. A process according to one of claims 1 to 4, characterized in that the reaction stream is derived from a process for amination/dehydration of an alkyl hydroxybenzoate consisting of reacting it with ammonia in the gas phase in the presence of a heterogeneous catalyst, preferably phosphoric acid deposited on a silica gel or kieselguhr type support.

9. A process according to one of claims 1 to 8, characterized in that the gaseous reaction stream is at a temperature of 200° C. to 600° C., preferably in the range 350° C. to 450° C.

10. A process according to claim 1, characterized in that it consists of heat treating the gas stream comprising 2-hydroxybenzonitrile completely or partially in its salt form.

11. A process according to claim 10, characterized in that the gaseous reaction stream is brought into contact with a solvent (organic solvent and/or water) then the ensemble is cooled to a temperature of less than 200° C., preferably in the range 200° C. to 100° C., to obtain a liquid phase essentially comprising 2-hydroxybenzonitrile.

12. A process according to claim 10 or claim 11, characterized in that the solvent (S1) is a polar or apolar solvent, preferably selected from: water, water/alcohol mixtures, preferably methanol or ethanol; halogenated or non halogenated aromatic hydrocarbons, preferably toluene, xylenes, ethylbenzene or monochlorobenzene; and ethers, preferably anisole or 2-ethylhexanol.

13. A process according to one of claims 10 to 12, characterized in that the hot gas stream is brought into contact with the vaporized reaction solvent generally as a co-current and in that light gases (F2) comprising ammonia, alcohol (usually methanol), water and in some cases an azeotropic water/organic solvent mixture are recovered, along with heavy compounds (F3), essentially 2-hydroxybenzonitrile and the solvent (organic solvent and/or water).

14. A process according to one of claims 10 to 13, characterized in that the heavy fraction (F3) essentially comprising 2-hydroxybenzonitrile and solvent is purified by distillation.

15. A process according to one of claims 10 to 13, characterized in that the heavy fraction (F3) essentially comprising 2-hydroxybenzonitrile is purified by crystallization.

16. A process according to claim 15, characterized in that the heavy fraction (F3) is cooled to a temperature in the range 100° C. to −10° C., preferably in the range 10° C. to 0° C., which causes the 2-hydroxybenzonitrile to crystallize; the crystalline product is then separated using conventional solid/liquid separation techniques, preferably by filtration; a solid is recovered that is essentially 2-hydroxybenzonitrile and a liquid phase (F6); and the solid is dried at a temperature in the range 30° C. to 80° C., preferably in the range 40° C. to 50° C.

17. A process according to one of claims 10 to 13, characterized in that the heavy fraction (F3) essentially comprising 2-hydroxybenzonitrile is purified by refining.

18. A process according to claim 17, characterized in that the heavy fraction 3) is cooled to a temperature in the range 100° C. to 30° C.; an aqueous phase (F7) and an organic phase (F7)3) which is essentially 2-hydroxybenzonitrile and solvent (S1) are separated; the organic phase is treated to separate the water and/or the solvent by distillation under reduced pressure; and a crude reaction mass essentially comprising 2-hydroxybenzonitrile is recovered which undergoes a purification operation by refining.

19. A process according to claim 18, characterized in that the reaction mass is cooled to a temperature in the range 10° C. to 50° C.; the temperature is raised to between 94° C. and 98° C. to separate eutectic fractions comprising impurities; and it is heated to a temperature of more than 98° C. to melt the 2-hydroxybenzonitrile, which is extracted in the molten state (F11).

20. A process according to claim 1, characterized in that it consists of liquefying the gaseous reaction stream then crystallizing the ammonium cyanophenate, then carrying out a solid/liquid separation and heat treating the solid product obtained.

21. A process according to claim 20, characterized in that firstly, the gaseous reaction stream (F1) is liquefied by cooling from 90° C.-100° C. to a temperature of 40° C.-30° C. and contact with water to eliminate light compounds and to recover an aqueous phase comprising ammonium cyanophenate and ammonia.

22. A process according to claim 20, characterized in that the liquefied stream (F12) is cooled to a temperature in the range 0° C.-30° C. to cause crystallization of the ammonium cyanophenate and the crystallized product is separated using conventional solid/liquid separation techniques, preferably by filtering to recover a solid which is essentially ammonium cyanophenate.

23. A process according to one of claims 20 to 22, characterized in that the heat treatment is then carried out at a temperature in the range 80° C. to 20° C., at a pressure in the range 1 mbar to 1 bar, preferably in the range 1 mbar to 500 mbar to recover the 2-hydroxybenzonitrile, in the solid form.

24. A process according to claim 1, characterized in that it consists of liquefying the gaseous reaction stream, then crystallizing the ammonium cyanophenate then carrying out solid/liquid separation to dissolve the solid product obtained in an organic solvent, preferably a polar solvent then heat treating the organic solution obtained.

25. A process according to claim 24, characterized in that it consists of liquefying the gaseous reaction stream then crystallizing the ammonium cyanophenate then carrying out solid/liquid separation as described in one of claims 21 to 23.

26. A process according to claim 24, characterized in that the solid product obtained is dissolved in an organic solvent, preferably a polar aprotic solvent, more preferably dimethylformamide or N-methylpyrrolidone.

27. A process according to claim 26, characterized in that the organic ammonium cyanophenate solution undergoes a heat treatment at a temperature in the range 80° C. to 20° C., at a pressure in the range 1 mbar to 1 bar, preferably in the range 1 mbar to 500 mbar, to eliminate a light fraction (F16) and to recover (F17), which is 2-hydroxybenzonitrile in solution in a solvent (S2).

28. A process according to claim 1, characterized in that it consists of liquefying the gaseous reaction stream, treating it with an acid to obtain 2-hydroxybenzonitrile, and carrying out liquid/liquid separation to recover 2-hydroxybenzonitrile in the organic phase.

29. A process according to claim 28, characterized in that the gaseous reaction stream (F1) is liquefied by cooling from 90° C.-100° C. to a temperature of 40° C.-30° C. and bringing it into contact with water to eliminate the light compounds and to recover an aqueous phase comprising ammonium cyanophenate and ammonia.

30. A process according to claim 28, characterized in that the ammonium ions are neutralized using a Brönsted acid with a pKa, measured in water, of less than 6, preferably in the range −1 to 4.

31. A process according to claim 30, characterized in that the acid is sulphuric acid.

32. A process according to claim 30 or claim 31, characterized in that the quantity of acid employed is such that the pH obtained is in the range 6 to 1, preferably in the range 4 to 3.

33. A process according to claim 28, characterized in that the reaction mixture obtained is decanted to separate an aqueous phase (F19) comprising the excess acid and the ammonium salts and an organic phase (F20) comprising 2-hydroxybenzonitrile.

34. A process according to one of claims 20 to 33, characterized in that the 2-hydroxybenzonitrile is purified using the purification techniques defined in one of claims 14 to 19.

35. A facility for separating 2-hydroxybenzonitrile from a gaseous reaction stream comprising 2-hydroxybenzonitrile, in the salt form, characterized in that it comprises:
a first column (1) intended to bring the gaseous reaction stream and the solvent (water and/or organic solvent) into contact;
a second column (2) the inlet to which is connected to the bottom of column (1), said column (2) being intended to produce a light fraction comprising ammonia, water and an alcohol (preferably methanol) from the column head and from the column bottom, a heavy fraction essentially comprising 2-hydroxybenzonitrile, the solvent (water and/or organic solvent), traces of ammonia and minor quantities of secondary products.

36. A facility according to claim 35, characterized in that the first column (1) is a wash tower comprising means for admitting a gaseous reaction stream (F1) to be treated and means for admitting a solvent (S1), a vaporizing device (nozzles) and means for evacuating a heavy phase (liquid and/or gaseous) connected to a second column (2) which is supplied at mid-height, comprising cooling means (condenser) and provided in its upper portion with means for evacuating a gas phase comprising light compounds (F2) and in its lower portion, means for withdrawing a liquid phase (F3).

37. A facility according to claim 36, characterized in that the separation unit is completed by a purification unit which is a distillation unit comprising a distillation column (3) supplied with the heavy fraction (F3) deriving from the preceding column (2) and designed to obtain:
at the column head, different fractions at a pressure of 0.4 mbar:
light fractions with a boiling point of less than 40° C. at atmospheric pressure;
a fraction between 40° C. and 110° C. corresponding to phenol and to methyl salicylate;
a fraction at 110° C. corresponding to 2-hydroxybenzonitrile (F4);
and at the column bottom, different impurities (F5) with a boiling point of more than 110° C.; salicylamide, N-methylsalicylamide and S-triazine.

38. A facility according to claim 37, characterized in that the distillation column (3) has the following specifications:
number of theoretical stages: 5 to 40, preferably 10 to 30;
reflux ratio R in the range 1 to 10;
low residence time, preferably less than 30 minutes.

39. A facility according to claim 35, characterized in that the separation unit is completed by a purification unit which is a crystallization unit comprising:
a crystallization reaction (4) provided with means for admitting the fraction (F3) and a withdrawal device connected to the separation device (5) and provided with agitation means and a temperature regulating device;
a solid/liquid separation device (6), preferably an aspirator, agitated filter, filter-press, or multi-tube filter to recover a solid essentially constituted by 2-hydroxybenzonitrile and an aqueous phase (F6) comprising the solvent, a little 2-hydroxybenzonitrile (less than 5% by weight) and by-products;
optionally, a drying means (6), preferably a rotary drier or an agitated drier, to recover 2-hydroxybenzonitrile in the form of a powder (P).

40. A facility according to claim 35, characterized in that the separation unit is completed by a purification unit which is a refining unit comprising:
a cooling tower (7) provided with means for admitting the fraction (F3) and a withdrawal device connected to a separation device (8);
a liquid/liquid separation device (8), preferably a settler, to separate an aqueous phase (F7) from an organic phase essentially constituted by 2-hydroxybenzonitrile (F8);
a reactor (9) provided with means for admitting an organic phase and provided with agitation means, a temperature regulating means (hot/cold), a distillation column and provided with a reduced pressurization system to separate, by distillation (F9), the organic solvent and/or remaining water and to recover crude 2-hydroxybenzonitrile;
one or more refining devices, preferably of the draining sieve type to eliminate impurities (F10) and to recover a liquid fraction of 2-hydroxybenzonitrile (F11).

41. A facility for separating 2-hydroxybenzonitrile by physical treatment of ammonium cyanophenate comprising:
a wash tower (11) provided with a system for admitting gas (F1) and a device for introducing water via a pump that can recover a liquid phase (F12) comprising water, crude ammonium cyanophenate and ammonia from the bottom; which mixture is transferred to reactor (12);
a crystallization reactor (12) for the ammonium cyanophenate and provided with means for admitting the fraction (F12) and a withdrawal device to connect it to a separation device (13); said reactor being provided with agitation means and a temperature regulating means;
a solid/liquid separation device (13), preferably an aspirator, agitated filter, filter-press, multi-tube filter to recover a solid essentially constituted by ammonium cyanophenate and an aqueous phase (F13) comprising the solvent, a little 2-hydroxybenzonitrile (less than 5% by weight) and by-products;
drying means (14), preferably an agitated drier, in which it undergoes a temperature and pressure cycle to decompose the solid ammonium cyanophenate to 2-hydroxybenzonitrile, to recover 2-hydroxybenzonitrile in the form of a powder (P) while eliminating the solvent and remaining ammonia (F14).

42. A facility for separating 2-hydroxybenzonitrile by physical treatment of ammonium cyanophenate comprising:
a wash tower (11) provided with a system for admitting gas (F1) and a device for introducing water via a pump that can recover a liquid phase (F12) comprising water, crude ammonium cyanophenate and ammonia from the bottom; which mixture is transferred to a reactor (12);
a crystallization reactor (12) for the ammonium cyanophenate and provided with means for admitting the fraction (F12) and a withdrawal device to connect it to a separation device (13); said reactor being provided with agitation means and a temperature regulating means;
a solid/liquid separation device (13), preferably an aspirator, agitated filter, filter-press, multi-tube filter to recover a solid essentially constituted by ammonium cyanophenate and an aqueous phase (F13) comprising the solvent, a little 2-hydroxybenzonitrile (less than 5% by weight) and by-products and provided with means for admitting a solvent (S2) to dissolve the ammonium cyanophenate;
a reactor (16) provided with a device for admitting an organic solution of ammonium cyanophenate provided with agitation means, a temperature regulating device surmounted by a distillation column and provided with a reduced pressurization system to separate, by distillation, the organic solvent and/or remaining water (F16) and to carry out thermal decomposition of ammonium cyanophenate to 2-hydroxybenzonitrile.

43. A facility for separating 2-hydroxybenzonitrile by chemical treatment of a gaseous reaction stream comprising 2-hydroxybenzonitrile in the salt form, characterized in that it comprises:
a wash tower (17) provided with a system for admitting gas (F1) and a device for introducing water via a pump that can recover a liquid phase (F18) comprising water, crude ammonium cyanophenate and ammonia from the bottom; which mixture is transferred to a reactor (18);
a neutralization reactor (18) provided with agitation means, a temperature regulating device, a device for introducing reactant (acid) and a pH regulating device (electrode): the lower portion of said reactor comprising a withdrawal device that can connect it to a device (19);
a liquid/liquid separation device (13), preferably a settler for separating an aqueous phase (F19) comprising excess acid and ammonium salts and an organic phase (F20) comprising 2-hydroxybenzonitrile.

44. A facility according to claims 41 to 43, characterized in that it is completed by a purification unit described in one of claims 37 to 40.

45. A hydroxybenzonitrile type compound in the form of an ammonium salt with formula (III): 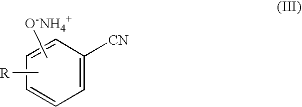
in which formula, R has the meanings given in claim 2 or claim 3.

46. Ammonium 2-cyanophenate with formula: 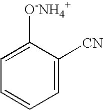
a liquid/liquid separation device (8), preferably a settler, to separate an aqueous phase (F7) from an organic phase essentially constituted by 2-hydroxybenzonitrile (F8);
a reactor (9) provided with means for admitting an organic phase and provided with agitation means, a temperature regulating means (hot/cold), a distillation column and provided with a reduced pressurization system to separate, by distillation (F9), the organic solvent and/or remaining water and to recover crude 2-hydroxybenzonitrile; and
one or more refining devices, preferably of the draining sieve type to eliminate impurities (F10) and to recover a liquid fraction of 2-hydroxybenzonitrile (F11).

88. A facility for separating 2-hydroxybenzonitrile by physical treatment of ammonium cyanophenate comprising:
a wash tower (11) provided with a system for admitting gas (F1) and a device for introducing water via a pump that can recover a liquid phase (F12) comprising water, crude ammonium cyanophenate and ammonia from the bottom; which mixture is transferred to reactor (12);
a crystallization reactor (12) for the ammonium cyanophenate and provided with means for admitting the fraction (F12) and a withdrawal device to connect it to a separation device (13); said reactor being provided with agitation means and a temperature regulating means;
a solid/liquid separation device (13), agitated filter, filter-press, multi-tube filter to recover a solid essentially constituted by ammonium cyanophenate and an aqueous
